(19) United States
(12) Patent Application Publication
(10) Pub. No.: US 2026/0334908 A1
(43) Pub. Date: Oct. 29, 2026

(54) REMOTE RADIOGRAPHY SYSTEM AND METHODS FOR SECURE, CLOUD-BASED X-RAY IMAGING

(71) Applicant: TensileX, Inc., Naples, FL (US)

(72) Inventors: Luca Bogoni, Philadelphia, PA (US); Giovanni Lorenz, San Antonio, TX (US)

(21) Appl. No.: 19/655,833

(22) Filed: Apr. 23, 2026

Related U.S. Application Data
(60) Provisional application No. 63/794,128, filed on Apr. 24, 2025.

(51) Int. Cl. A61B 6/00; A61B 6/46

(52) U.S. Cl. CPC A61B 6/548; A61B 6/46; A61B 6/542

(57) ABSTRACT

A remote radiography architecture connects an imaging site to an orchestration and governance component and a remote operator interface across structured and unstructured deployments, including fixed x-ray rooms and portable or home installations. An on-site integration system couples a cloud layer to an OEM imaging system and limits site-side interaction to positioning, communications, calibration when applicable, and readiness tasks while exposure authorization remains with a remote operator. Device status and current imaging parameters, including site-established acquisition parameters, are relayed to the remote operator before an exposure command is released. Optional views, including a camera integrated into the OEM imaging system when available, phantom-based calibration when appropriate, OEM-defined interface translation, and multi-network continuity support distributed deployment. A network quality gate evaluates communication indicators, enforces a stabilization period before pre-exposure authorization, and records event history in an immutable event store, and optional AI-enabled tools support review of image quality or protocol compliance.

### CROSS-REFERENCE TO RELATED APPLICATIONS

This application claims the benefit of priority to a U.S. provisional patent application Ser. No. 63/794,128, filed on Apr. 24, 2025, and titled REMOTE RADIOGRAPHY SYSTEM AND METHODS FOR SECURE, CLOUD-BASED X-RAY IMAGING, which is hereby incorporated by reference herein in its entirety.

### FIELD

The present disclosure relates generally to medical imaging systems and, more particularly, to remote radiography systems in which a remote operator controls an original equipment manufacturer (OEM) imaging system through a cloud-hosted orchestration and governance component while an on-site participant performs physical setup at an imaging site.

### BACKGROUND

Radiographic imaging remains a core diagnostic tool for pulmonary, thoracic, orthopedic, and bedside evaluation. Conventional workflows generally place a licensed radiographer at the patient location so that the licensed radiographer can set up equipment, position the patient, choose exposure settings, confirm readiness, and trigger the exposure.

Conventional workflows can become difficult when imaging support is needed across a range of deployment types, including hospital x-ray rooms, clinics, rural locations, assisted-living environments, residences, satellite sites, and temporary or low-volume sites. Portable equipment can address transport of hardware for some deployments, but transport of hardware alone does not resolve lawful control of ionizing-radiation equipment, whether the OEM imaging system is transported to the site or already installed at the site, when the physically present person is not the licensed radiographer who issues exposure authority.

Remote support arrangements can also become unreliable when communications vary in quality, when an OEM imaging system uses vendor-specific interfaces, when calibration should be confirmed after transport or infrequent use, or when the person at the imaging site has only setup responsibilities. A bare remote-trigger path can leave unresolved questions regarding readiness confirmation, current imaging parameter information, event logging, or transition between a primary communication path and a fallback communication path.

Systems designed only for fixed radiography rooms, permanent enclosures, or dedicated kiosks may not translate well to home deployment, retrofit operation, satellite-site operation, or other geographically distributed settings in which an on-site participant performs setup while exposure authority remains remote. Conversely, systems designed only for transported or temporary-site operation may not fully account for structured acquisition environments such as hospital x-ray rooms or established clinic rooms. A need therefore exists for a remote radiography architecture that separates physical setup from exposure authority while still supporting calibration when appropriate, OEM integration, readiness review, safety gating, and communication continuity.

### SUMMARY

In one aspect, a method of remotely controlling a radiographic examination includes authenticating, at a cloud-hosted orchestration and governance component, a remote operator for a control session associated with an OEM imaging system located at an imaging site; receiving, at the cloud-hosted orchestration and governance component and from an on-site integration system coupled to the OEM imaging system, device status information and current imaging parameter information representing a readiness state of the OEM imaging system; presenting, by a remote operator interface coupled to the cloud-hosted orchestration and governance component, the device status information and the current imaging parameter information to the remote operator; receiving, at the cloud-hosted orchestration and governance component, an exposure authorization from the remote operator; transmitting, responsive to the exposure authorization and via a secure network connection through the on-site integration system, an exposure command to the OEM imaging system; and receiving, via the secure network connection, a radiographic image captured by the OEM imaging system, wherein an on-site participant physically positions the OEM imaging system at the imaging site, and wherein an on-site interaction interface available at the imaging site lacks authority to initiate emission of X-ray radiation.

In some embodiments, the current imaging parameter information includes acquisition parameter information established at the imaging site, the acquisition parameter information including one or more of source-to-image distance information, geometry information, mAs information, tube-voltage information, or body-size information. In some embodiments, the on-site interaction interface presents guided instructions or communications for positioning the OEM imaging system, performing a calibration routine, or otherwise coordinating the radiographic examination, the communications including at least one of voice, video, or chat. In some embodiments, the method includes directing placement of a calibration phantom at the imaging site and receiving a test exposure generated with the calibration phantom before patient imaging.

In some embodiments, the method includes receiving, at the cloud-hosted orchestration and governance component, image data from at least one of a wide-field camera, a narrow-field camera, or a camera integrated into the OEM imaging system when available for alignment review by the remote operator. In some embodiments, the method includes writing an immutable event record to an immutable event store, the immutable event record representing at least one of the device status information, the current imaging parameter information, or the exposure authorization. In some embodiments, the method includes comparing a patient identifier and a study identifier associated with the control session to a scheduled order and withholding the exposure authorization when a mismatch is detected.

In another aspect, a distributed radiography orchestration system includes a cloud-hosted orchestration and governance component; a remote operator interface communicatively coupled to the cloud-hosted orchestration and governance component and configured to present device status information and current imaging parameter information to a remote operator; an on-site integration system configured to couple the cloud-hosted orchestration and governance component to an OEM imaging system at an imaging site, the on-site integration system including an OEM abstraction layer configured to exchange command or status transactions with the OEM imaging system through an OEM-defined interface; and an on-site interaction interface configured to assist an on-site participant with physical setup of the OEM imaging system, wherein the cloud-hosted orchestration and governance component is configured to issue an exposure authorization for the OEM imaging system only after receiving an exposure authorization from the remote operator, and wherein the on-site interaction interface lacks authority to initiate emission of X-ray radiation.

In some embodiments, the on-site integration system includes a secure communication endpoint configured to establish an encrypted communication session with the cloud-hosted orchestration and governance component. In some embodiments, the OEM abstraction layer is configured to translate a cloud-originated command into an OEM-defined interface transaction for the OEM imaging system. In some embodiments, the on-site integration system includes a local execution interface mechanism including at least one of a digital interface, a wireless accessory interface, a physical hand-switch interface, or an intermediary hardware adapter.

In some embodiments, the on-site integration system includes an image and status relay and a local state reporter configured to transmit the device status information and the current imaging parameter information from the OEM imaging system to the cloud-hosted orchestration and governance component. In some embodiments, the on-site integration system includes a camera interface module configured to receive image data from at least one of a wide-field camera, a narrow-field camera, or a camera integrated into the OEM imaging system when available. In some embodiments, the remote operator interface is configured to present concurrent session status for a plurality of imaging sites.

In a further aspect, a distributed radiography exposure-authorization system includes a cloud-hosted orchestration and governance component; an on-site integration system communicatively coupled to an OEM imaging system; a network quality gate communicatively coupled between the cloud-hosted orchestration and governance component and the on-site integration system; and an immutable event store storing immutable event records, wherein the network quality gate is configured to receive objective system-measured indicators for a communication path between the cloud-hosted orchestration and governance component and the on-site integration system, determine a communication-quality state from the objective system-measured indicators, enforce a stabilization period before release of a pre-exposure authorization to the on-site integration system, write at least one of the immutable event records to the immutable event store, and withhold the pre-exposure authorization while the communication-quality state fails an authorization condition.

In some embodiments, the on-site integration system is configured to transition from a primary communication interface to a fallback communication interface while the network quality gate withholds the pre-exposure authorization until completion of the stabilization period. In some embodiments, the objective system-measured indicators comprise at least one of latency values, jitter values, packet-loss values, or connection-continuity values for the communication path. In some embodiments, the immutable event records store the communication-quality state, the authorization condition, and issuance or withholding of the pre-exposure authorization.

In some embodiments, the on-site integration system is configured to block the pre-exposure authorization when an on-site participant proximity measurement indicates that an on-site participant is within a local safety boundary around the OEM imaging system. In some embodiments, the network quality gate is limited to pre-exposure authorization and does not participate in radiation termination after initiation of an authorized exposure.

### BRIEF DESCRIPTION OF THE DRAWINGS

FIG. 1 is a schematic architectural view of a distributed radiography orchestration system showing an imaging site, a cloud-hosted orchestration and governance component, and a remote operator environment.

FIG. 2 is a layered interaction view showing patient-location components, camera support, communication paths, and operator-side control relationships for the distributed radiography orchestration system of FIG. 1.

FIG. 3 is a block diagram of an on-site integration system that couples the cloud-hosted orchestration and governance component to an OEM imaging system through an OEM-defined interface.

FIG. 4 is a state-oriented diagram showing a network quality gate, objective system-measured indicators, a communication-quality state, a stabilization period, and immutable event logging used before release of a pre-exposure authorization.

FIG. 5 is a flow diagram of a remote examination control method including remote-operator authentication, status and current-parameter review, remote exposure authorization, an execution-constraint check evaluating system-level conditions before command transmission, transmission of an exposure command through the on-site integration system, and receipt of a radiographic image.

FIG. 6 is a flow diagram of a calibration and readiness-confirmation workflow including calibration determination, optional phantom verification, device-status and current-parameter review, withholding of progression when a readiness issue is present, and release when the readiness issue is cleared.

FIG. 7 is a flow diagram of a patient-study verification and local safety workflow including receipt of patient and study identifiers, comparison to a scheduled order, local safety review, withholding of pre-exposure authorization when a mismatch or unsafe condition is present, and permitting progression when verification and safety conditions are satisfied.

FIG. 8 is a flow diagram of a communication fallback and authorization-control workflow including evaluation of a primary communication interface, transition to a fallback communication interface after inadequate connectivity is detected, enforcement of a stabilization period, withholding of pre-exposure authorization while an authorization condition remains unsatisfied, and release of pre-exposure authorization after the authorization condition is satisfied.

### DETAILED DESCRIPTION OF EXAMPLE EMBODIMENTS

The disclosed subject matter can be embodied as a distributed radiography orchestration system 100 that separates physical setup at an imaging site 102 from exposure authority maintained by a remote operator 122 through a cloud-hosted orchestration and governance component 104. The distributed radiography orchestration system 100 can be used with a portable deployment, a home deployment, a retrofit deployment, an assisted-living deployment, a small-clinic deployment, a hospital x-ray-room deployment, a satellite-site deployment, or another geographically distributed deployment, thereby spanning structured acquisition environments and unstructured deployments in which an OEM imaging system 108 is placed at the imaging site 102 while a remote operator 122 remains away from the imaging site 102.

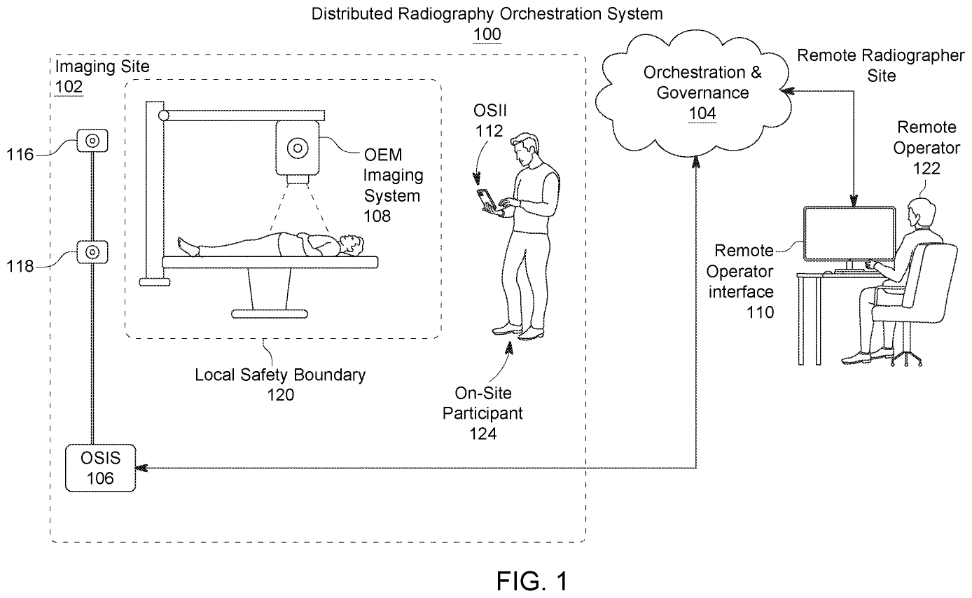

Referring to FIG. 1, the distributed radiography orchestration system 100 includes the imaging site 102, the cloud-hosted orchestration and governance component 104, the on-site integration system 106, the OEM imaging system 108, the remote operator interface 110, the on-site interaction interface 112, the wide-field camera 116, the narrow-field camera 118, the local safety boundary 120, the remote operator 122, and the on-site participant 124. The imaging site 102 can be a residence, a bedside environment, an assisted-living room, a clinic room, a hospital x-ray room, or another temporary or fixed location at which the OEM imaging system 108 is physically arranged for radiographic imaging.

The cloud-hosted orchestration and governance component 104 can authenticate the remote operator 122 for a control session associated with the OEM imaging system 108. The cloud-hosted orchestration and governance component 104 can also receive device status information and current imaging parameter information from the on-site integration system 106 and can present the device status information and the current imaging parameter information to the remote operator 122 through the remote operator interface 110 before release of an exposure authorization.

The remote operator interface 110 can present a session view that includes live video, still images, device status information, current imaging parameter information, or event history for the OEM imaging system 108. In some embodiments, the session view can switch among live-video, still-image, or bandwidth-managed displays while preserving operator access to readiness state and acquisition parameters. The remote operator interface 110 can also receive an exposure authorization from the remote operator 122 and deliver the exposure authorization to the cloud-hosted orchestration and governance component 104. The remote operator 122 can be a licensed radiographer or another credentialed operator authorized for the jurisdiction or workflow in use.

In certain embodiments, the exposure authorization provided by the remote operator may be subject to additional validation or execution constraints applied by the cloud-hosted orchestration and governance component prior to transmission of an exposure command.

The on-site participant 124 physically positions the OEM imaging system 108 at the imaging site 102 while the on-site interaction interface 112 lacks authority to initiate emission of X-ray radiation. The on-site interaction interface 112 can assist the on-site participant 124 with unpacking, setup, patient positioning, detector placement, source positioning, calibration prompts when applicable, communication with the remote operator 122, and recovery prompts. The on-site interaction interface 112 can therefore support the physical tasks that occur at the imaging site 102 without transferring exposure authority away from the remote operator 122. In some embodiments, including structured x-ray-room deployments, the workflow can begin after system installation and can involve only a subset of the illustrated tasks, such as patient positioning, detector positioning, or source positioning.

The local safety boundary 120 can define a region around the OEM imaging system 108 within which the on-site participant 124 should not remain during an authorized exposure. The local safety boundary 120 can be implemented by wearable tracking, radio-frequency location tracking, infrared markers, optical monitoring, line-of-sight prompts, or other proximity techniques. In structured environments such as an x-ray room, the local safety boundary 120 can additionally correspond to a shielded area or another preexisting safety control. The on-site integration system 106 can block a pre-exposure authorization when an on-site participant proximity measurement indicates that the on-site participant 124 is within the local safety boundary 120 around the OEM imaging system 108.

The wide-field camera 116 and the narrow-field camera 118 can capture image data for alignment review by the remote operator 122. The wide-field camera 116 can provide room-context imaging, overall patient-location context, or gross placement review. The narrow-field camera 118 can provide closer imaging of detector position, source orientation, patient anatomy alignment, or other setup details. The cloud-hosted orchestration and governance component 104 can receive image data from at least one of the wide-field camera 116 or the narrow-field camera 118 for alignment review before release of an exposure authorization.

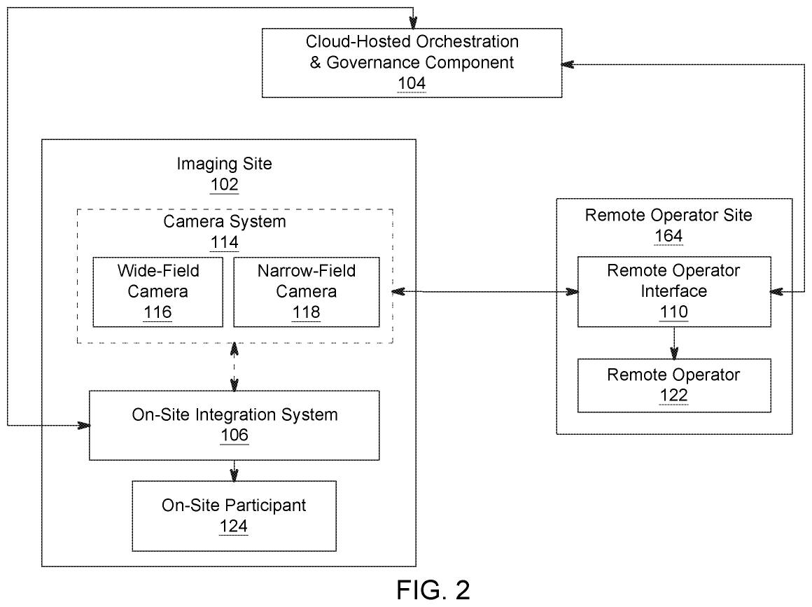

Turning to FIG. 2, a camera system 114 includes the wide-field camera 116 and the narrow-field camera 118, and the camera system 114 cooperates with the on-site integration system 106 and the remote operator interface 110 to distinguish among physical interactions, secure communications, and data transfer paths. The FIG. 2 arrangement emphasizes that the patient-location environment can include direct physical activity by the on-site participant 124 while control traffic and image transfer remain associated with the cloud-hosted orchestration and governance component 104 and the remote operator interface 110.

The remote operator site 164 can include the remote operator interface 110 and the remote operator 122 in a location remote from the imaging site 102. The remote operator interface 110 can present device status information and current imaging parameter information for one control session or for concurrent control sessions associated with a plurality of imaging sites 102. The remote operator interface 110 can therefore present concurrent session status for multiple imaging sites 102 while the cloud-hosted orchestration and governance component 104 maintains separate exposure-authority logic for each control session.

The camera system 114 can support a live-video mode, a still-image mode, or a bandwidth-managed hybrid mode, and can receive a feed from a camera integrated into the OEM imaging system 108 when available. In a higher-bandwidth condition, the camera system 114 can deliver live video from the imaging site 102 to the remote operator interface 110. In a lower-bandwidth condition, the camera system 114 can deliver periodic still images or compressed video while the cloud-hosted orchestration and governance component 104 continues to receive readiness information and current imaging parameter information from the on-site integration system 106. The camera system 114 or the immutable event store 128 can also preserve selected still images or video segments as documentation confirming patient positioning or clearance of the local safety boundary 120 at the time of an authorized exposure.

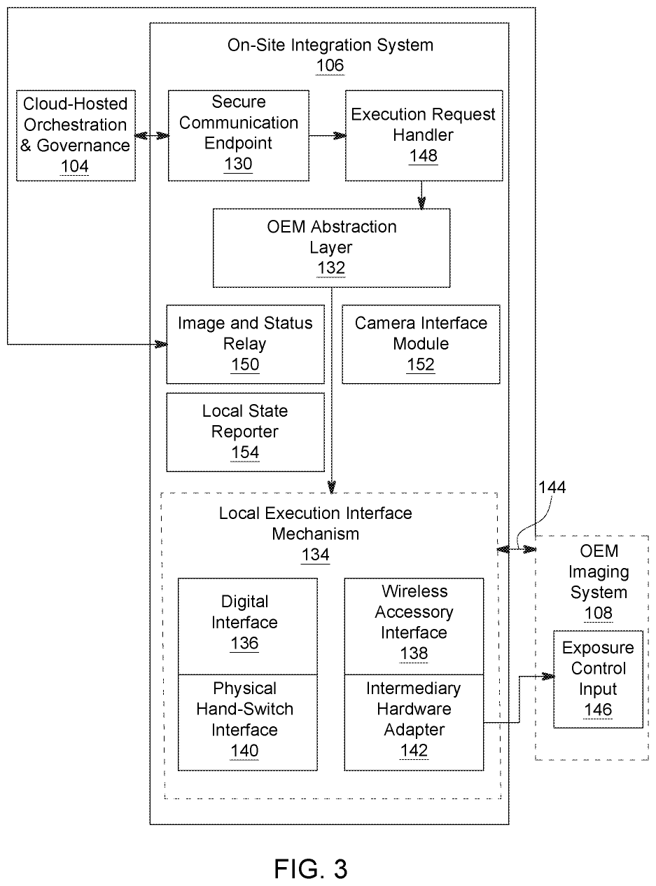

As shown in FIG. 3, the on-site integration system 106 includes the secure communication endpoint 130, the execution request handler 148, the OEM abstraction layer 132, the image and status relay 150, the camera interface module 152, the local state reporter 154, the local execution interface mechanism 134, the digital interface 136, the wireless accessory interface 138, the physical hand-switch interface 140, the intermediary hardware adapter 142, the OEM-defined interface 144, and the exposure control input 146 associated with the OEM imaging system 108. The on-site integration system 106 can therefore function as a vendor-neutral gateway between the cloud-hosted orchestration and governance component 104 and the OEM imaging system 108.

The secure communication endpoint 130 can establish an encrypted communication session with the cloud-hosted orchestration and governance component 104. The secure communication endpoint 130 can implement a primary communication interface and a fallback communication interface. The primary communication interface can use a local wireless network, a wired network, or another preferred path, and the fallback communication interface can use a cellular path or another secondary path. During transition from the primary communication interface to the fallback communication interface, the secure communication endpoint 130 and the network quality gate 126 can continue to withhold a pre-exposure authorization until communication conditions satisfy an authorization condition after a stabilization period.

The execution request handler 148 can receive a cloud-originated command associated with an exposure authorization, a calibration request, a setup request, or a status request. The execution request handler 148 can coordinate forwarding of the cloud-originated command to the OEM abstraction layer 132 and can prevent local setup activity from being interpreted as exposure authority. The execution request handler 148 can also coordinate issuance of a command to the OEM-defined interface 144 only after the cloud-hosted orchestration and governance component 104 has released exposure authority for the control session.

The OEM abstraction layer 132 can exchange command or status transactions with the OEM imaging system 108 through the OEM-defined interface 144. The OEM abstraction layer 132 can translate a cloud-originated command into an OEM-defined interface transaction for the OEM imaging system 108 and can translate OEM-specific status information into a normalized status representation for the cloud-hosted orchestration and governance component 104. The OEM abstraction layer 132 can therefore support retrofit operation with more than one OEM imaging system 108 without forcing the cloud-hosted orchestration and governance component 104 to rely on a single source-specific command set.

The local execution interface mechanism 134 can include at least one of the digital interface 136, the wireless accessory interface 138, the physical hand-switch interface 140, or the intermediary hardware adapter 142. The digital interface 136 can support a wired or digital control path into the OEM-defined interface 144. The wireless accessory interface 138 can support connection to a wireless accessory or peripheral device associated with the OEM imaging system 108. The physical hand-switch interface 140 can support installations in which a physical switch path remains present in the OEM imaging system 108 but is mediated by the on-site integration system 106. The intermediary hardware adapter 142 can support legacy or retrofit operation in which a direct OEM-defined interface 144 is unavailable.

The OEM-defined interface 144 can couple the local execution interface mechanism 134 to the OEM imaging system 108 and, more specifically, to the exposure control input 146 or another OEM command path. The OEM-defined interface 144 can be an application programming interface path, a protocol adapter path, a signal-conditioning path, a connector path, or another OEM-specific interface path. The exposure control input 146 can receive a translated authorization command from the on-site integration system 106 only after the cloud-hosted orchestration and governance component 104 and the network quality gate 126 permit release of a pre-exposure authorization.

The image and status relay 150 and the local state reporter 154 can transmit device status information, current imaging parameter information, and radiographic image data from the OEM imaging system 108 to the cloud-hosted orchestration and governance component 104. The device status information can include readiness state, battery status, detector availability, source status, thermal status, and fault information. The current imaging parameter information can include source-to-image distance information, geometry information, detector orientation information, protocol information, tube-voltage information, tube-current information, exposure-time information, or a subset of such information. The remote operator interface 110 can present the device status information and the current imaging parameter information to the remote operator 122 before release of an exposure authorization.

The camera interface module 152 can receive image data from the wide-field camera 116, the narrow-field camera 118, or another imaging sensor associated with the imaging site 102. The camera interface module 152 can compress, route, buffer, or prioritize image data according to available bandwidth while maintaining readiness reporting through the image and status relay 150 and the local state reporter 154. The camera interface module 152 can also support mixed operating modes in which the wide-field camera 116 sends lower-rate room-context imaging and the narrow-field camera 118 sends alignment-focused imaging during setup.

The on-site interaction interface 112 can present guided instructions or communications for positioning the OEM imaging system 108, performing a calibration routine when applicable, or coordinating readiness with the remote operator 122. The on-site interaction interface 112 can receive the guided instructions, voice communications, video communications, chat communications, corrective prompts, or workflow state information from the cloud-hosted orchestration and governance component 104 through the on-site integration system 106 or through a separate communication path to the cloud-hosted orchestration and governance component 104. The guided instructions or communications can be displayed or rendered on a dedicated local display, a tablet, a phone, or another consumer device at the imaging site 102. In some embodiments, the on-site interaction interface 112 can walk the on-site participant 124 through detector placement, source height selection, acquisition-parameter confirmation, patient posture, shielding placement, or recovery from an error condition reported by the local state reporter 154.

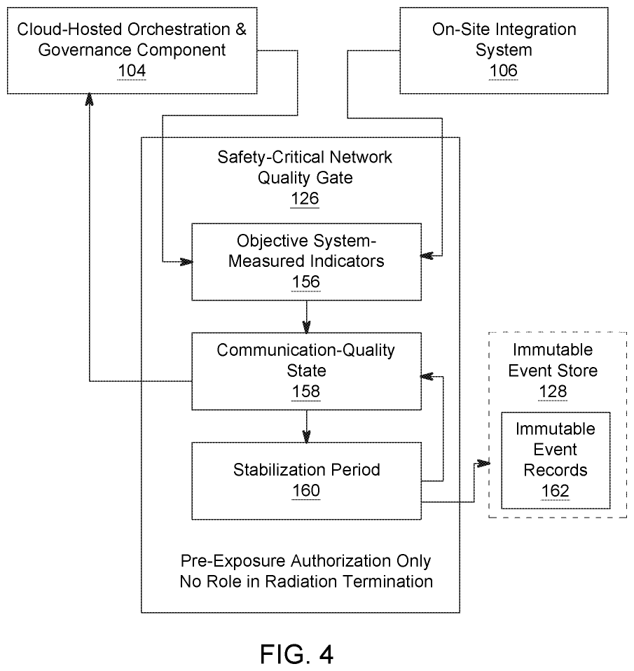

With reference to FIG. 4, the network quality gate 126 receives objective system-measured indicators 156 for a communication path between the cloud-hosted orchestration and governance component 104 and the on-site integration system 106, determines a communication-quality state 158 from the objective system-measured indicators 156, and enforces a stabilization period 160 before release of a pre-exposure authorization to the on-site integration system 106. The network quality gate 126 can therefore prevent issuance of a pre-exposure authorization during a degraded communication state, during a transition between communication interfaces, or during recovery immediately after restoration of communications.

The objective system-measured indicators 156 can include latency values, jitter values, packet-loss values, connection-continuity values, heartbeat continuity values, or comparable communication-health metrics. The communication-quality state 158 can represent a release-ready state, a degraded state, a recovering state, or another communication state used by the network quality gate 126. The stabilization period 160 can represent an interval during which the communication-quality state 158 remains satisfactory before release of a pre-exposure authorization.

The immutable event store 128 can store immutable event records 162 associated with the communication-quality state 158, the authorization condition, and issuance or withholding of a pre-exposure authorization. The immutable event records 162 can also store device status information, current imaging parameter information, network-transition events, calibration events, patient-verification events, or user-authorization events. The immutable event store 128 can therefore support troubleshooting, compliance review, quality review, and reconstruction of the communication state that existed before a patient exposure.

The network quality gate 126 can be limited to pre-exposure authorization and can remain outside the path used for radiation termination after initiation of an authorized exposure. In such embodiments, once the OEM imaging system 108 begins an authorized exposure, radiation termination can remain governed by local equipment logic, OEM safety logic, or another direct control path not dependent on continuing cloud release from the network quality gate 126. The pre-exposure-only role can reduce the risk that a transient network fault interferes with post-initiation radiation-termination behavior.

The pre-exposure authorization controlled by the network quality gate 126 and the exposure authorization granted by the remote operator 122 can operate as sequential prerequisites for release of an exposure command to the OEM imaging system 108. In some embodiments, the network quality gate 126 releases the pre-exposure authorization to the on-site integration system 106 before the remote operator 122 is permitted to issue the exposure authorization through the remote operator interface 110. In other embodiments, the exposure authorization from the remote operator 122 can be received at the cloud-hosted orchestration and governance component 104 while the network quality gate 126 independently evaluates the communication-quality state 158, and the exposure command is transmitted to the OEM imaging system 108 only after both the pre-exposure authorization and the exposure authorization have been obtained.

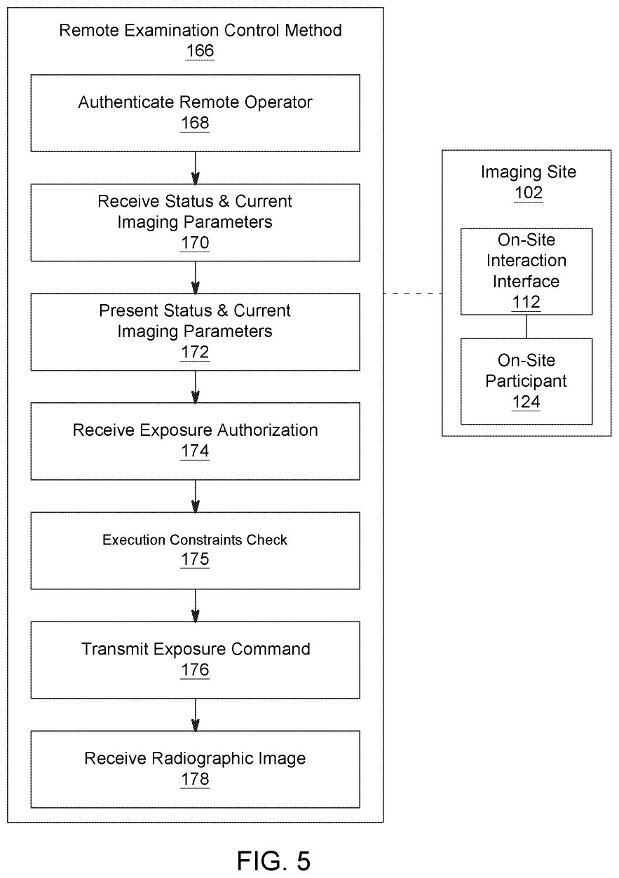

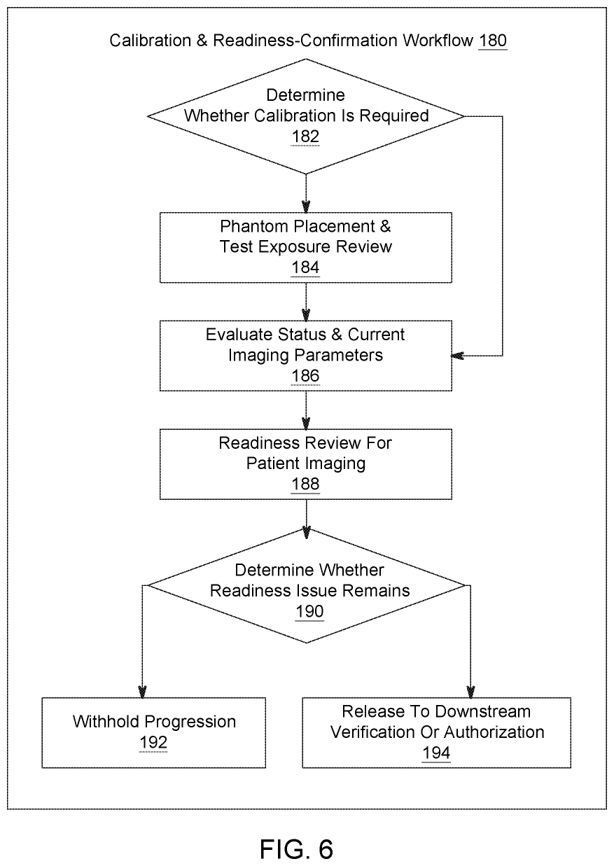

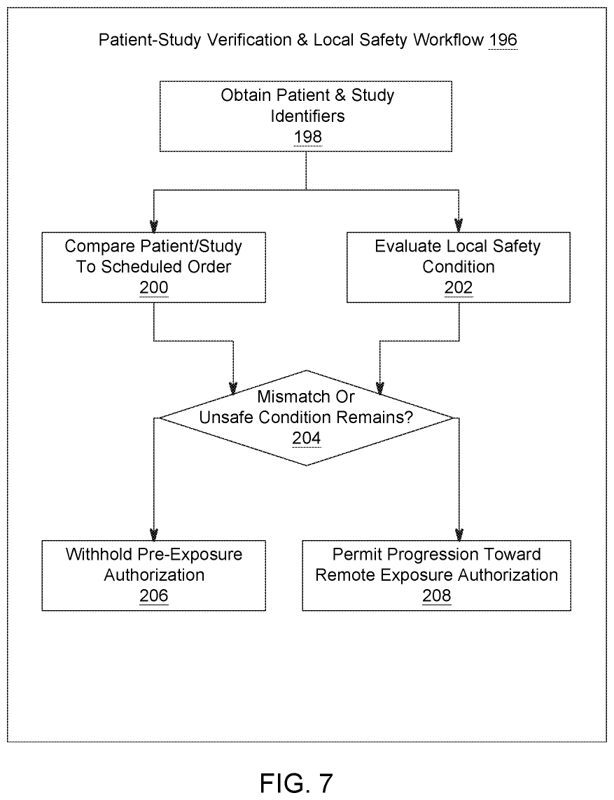

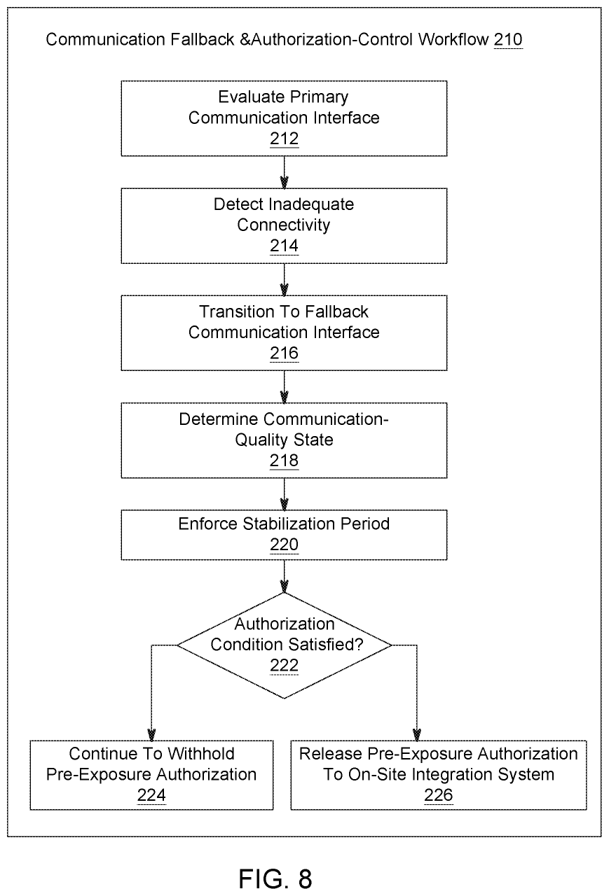

FIG. 5 illustrates a remote examination control method 166. At an authentication step 168, the cloud-hosted orchestration and governance component 104 authenticates the remote operator 122 for a control session associated with the OEM imaging system 108 at the imaging site 102. At a status-receipt step 170, the cloud-hosted orchestration and governance component 104 receives device status information and current imaging parameter information from the on-site integration system 106. At a presentation step 172, the remote operator interface 110 presents the device status information and the current imaging parameter information to the remote operator 122. At an authorization-receipt step 174, the cloud-hosted orchestration and governance component 104 receives an exposure authorization input from the remote operator 122 after review of the reported readiness state and operational conditions. At an execution-constraint-check step 175, the cloud-hosted orchestration and governance component 104 evaluates one or more system-level execution conditions, including at least one of network quality, system state, or environmental validation, before permitting transmission of the exposure command. At an exposure-command step 176, an exposure command is transmitted through a secure network connection via the on-site integration system 106 to the OEM imaging system 108. Said exposure command is responsive to the exposure authorization input and subject to system-level execution conditions. At an image-receipt step 178, a radiographic image captured by the OEM imaging system 108 is received for review by the remote operator 122. Throughout the method 166, the on-site participant 124 performs physical positioning and setup through the on-site interaction interface 112 while the on-site interaction interface 112 lacks authority to initiate emission of X-ray radiation. The calibration and readiness-confirmation workflow 180 of FIG. 6, the patient-study verification and local safety workflow 196 of FIG. 7, and the communication fallback and authorization-control workflow 210 of FIG. 8 can each be executed as part of or in conjunction with the remote examination control method 166, for example before the authorization-input step 174 or the exposure-command step 176, so that the readiness, verification, and communication conditions addressed by those workflows are satisfied before the exposure command reaches the OEM imaging system 108.

In the embodiment illustrated in FIG. 5, exposure authorization is provided by the remote operator 122 based on evaluation of system readiness, environmental conditions, and communication status as presented through the remote operator interface. The cloud-hosted orchestration and governance component 104 acts to convey the resulting exposure command to the OEM imaging system 108 and may enforce execution constraints at the moment of command transmission.

In alternative embodiments, the exposure authorization input from the remote operator may be treated as one of a plurality of inputs evaluated by the cloud-hosted orchestration and governance component 104, which may further determine whether exposure authorization conditions are satisfied based on system state, environmental validation, and communication integrity prior to permitting execution. In such embodiments, exposure is permitted only when the combined conditions are satisfied, while preserving separation between authorization, execution, and on-site interaction.

FIG. 6 illustrates a calibration and readiness-confirmation workflow 180 that can be executed before patient exposure in deployments for which calibration or pre-exposure verification is appropriate. The workflow 180 is one example and, depending on deployment type, installed system state, or device condition, one or more illustrated steps can be omitted, repeated, or reordered. At a calibration-determination step 182, the distributed radiography orchestration system 100 determines whether a calibration phantom or another verification object should be placed at the imaging site 102 and whether a test exposure should be reviewed before patient imaging. At a phantom-verification step 184, the on-site participant 124 positions the calibration phantom and the remote operator 122 receives a resulting test exposure when calibration is required, and the phantom-verification step 184 can be omitted for an installed x-ray-room deployment or another already-calibrated configuration. At a parameter-review step 186, the on-site integration system 106 and the cloud-hosted orchestration and governance component 104 evaluate device status information and current imaging parameter information associated with the OEM imaging system 108. At a readiness-review step 188, the distributed radiography orchestration system 100 determines whether the OEM imaging system 108 satisfies readiness conditions for patient imaging. At a readiness-hold evaluation step 190, the distributed radiography orchestration system 100 determines whether any readiness issue remains present, including an unsatisfactory calibration result, an out-of-range readiness condition, or another setup issue that should be corrected before patient exposure. When a readiness issue remains present, a readiness-withhold step 192 withholds progression and can issue a corrective prompt through the remote operator interface 110 or the on-site interaction interface 112. When the readiness issue has been cleared, a readiness-release step 194 permits progression to downstream verification or remote exposure authorization for the control session.

FIG. 7 illustrates a patient-study verification and local safety workflow 196 that can be executed before release of a pre-exposure authorization. At a verification-intake step 198, session identifiers including a patient identifier and a study identifier are obtained for the control session. At an order-comparison step 200, the patient identifier and the study identifier are compared to a scheduled order, a radiology information system entry, or another scheduling source. At a safety-condition step 202, the distributed radiography orchestration system 100 evaluates whether the on-site participant 124 is outside the local safety boundary 120 and whether other local safety conditions have been satisfied. At a mismatch evaluation step 204, the distributed radiography orchestration system 100 determines whether a patient-order mismatch or an unsafe local condition remains present. When a mismatch or unsafe condition remains present, a verification-withhold step 206 withholds a pre-exposure authorization and can issue a corrective prompt through the remote operator interface 110 or the on-site interaction interface 112. When the patient-study association is verified and the safety condition has been cleared, a verification-release step 208 permits progression toward remote exposure authorization for the control session.

FIG. 8 illustrates a communication fallback and authorization-control workflow 210 associated with the network quality gate 126 and the secure communication endpoint 130. At a primary-link evaluation step 212, the distributed radiography orchestration system 100 evaluates a primary communication interface for a control session. At a connectivity-detection step 214, the network quality gate 126 or the secure communication endpoint 130 detects inadequate connectivity based on objective system-measured indicators 156. At a fallback-transition step 216, the secure communication endpoint 130 transitions from the primary communication interface to a fallback communication interface when the inadequate connectivity condition persists. At a communication-state determination step 218, the network quality gate 126 determines a communication-quality state 158 from the objective system-measured indicators 156 after the transition. At a stabilization-enforcement step 220, the network quality gate 126 enforces the stabilization period 160 before release of a pre-exposure authorization. At an authorization-condition evaluation step 222, the distributed radiography orchestration system 100 determines whether the communication-quality state 158 satisfies the authorization condition for release of a pre-exposure authorization. When the authorization condition remains unsatisfied, an authorization-withhold step 224 continues to withhold the pre-exposure authorization. When the authorization condition has been satisfied after the stabilization period 160, an authorization-release step 226 releases the pre-exposure authorization to the on-site integration system 106.

The distributed radiography orchestration system 100 can support a calibration workflow in which the on-site participant 124 places a calibration phantom at the imaging site 102 and the remote operator 122 receives a test exposure generated with the calibration phantom before patient imaging. The remote operator interface 110 can present the resulting test exposure, device status information, and current imaging parameter information so that the remote operator 122 can confirm readiness before authorizing patient exposure. The calibration workflow can be used after shipment, after detector replacement, after source replacement, after an extended idle period, or after any transport event that could alter geometry or alignment.

The current imaging parameter information can include clinical imaging protocol, acquisition parameter information established at the imaging site 102. The acquisition parameter information can therefore include source-to-image distance information, geometry information, mAs information, tube-voltage information, body-size information, or other values that originate from a local sensor, a local measurement path, or physical setup activity at the imaging site 102 rather than only from direct remote entry by the remote operator 122. The cloud-hosted orchestration and governance component 104 can still present those site-established values to the remote operator 122 so that the remote operator 122 can confirm the actual physical arrangement and acquisition configuration that exist at the imaging site 102 before authorizing exposure.

The distributed radiography orchestration system 100 can also compare a patient identifier and a study identifier associated with the control session to a scheduled order and can withhold the exposure authorization when a mismatch is detected. The patient identifier can be acquired by manual entry, barcode entry, bracelet scanning, image capture, or another patient-verification technique. The study identifier can be derived from a scheduled order, a radiology information system, or another scheduling source. The cloud-hosted orchestration and governance component 104 can withhold a pre-exposure authorization until the patient identifier and the study identifier align with the scheduled order. In some embodiments, the on-site integration system 106 can independently verify the patient identifier and the study identifier against the scheduled order and can withhold the pre-exposure authorization at the on-site integration system 106 when a mismatch is detected.

In a home deployment, the imaging site 102 can be a residence at which the on-site participant 124 is a family member, a caregiver, a visiting aide, or another person who does not hold exposure authority for the OEM imaging system 108. The on-site interaction interface 112 can provide a simplified workflow for unpacking a portable detector, placing the detector under or behind a patient, positioning a source support, confirming source-to-image distance information, and standing outside the local safety boundary 120. The cloud-hosted orchestration and governance component 104 can maintain control over readiness review and exposure authorization throughout the home deployment.

In an assisted-living deployment, a small-clinic deployment, or a hospital x-ray-room deployment, the on-site participant 124 can have partial clinical training while still lacking exposure authority for the OEM imaging system 108. The on-site integration system 106 can therefore support a shorter setup workflow than a consumer-directed workflow while still maintaining remote-only exposure authorization. In a structured x-ray-room deployment, the OEM imaging system 108 can already be installed and the on-site participant 124 can operate within a shielded area or other established room-safety arrangement while the remote operator 122 retains exposure authority. The same architecture can be used when the OEM imaging system 108 is shipped to the site, when the OEM imaging system 108 is already present at the site, or when the intermediary hardware adapter 142 is used to retrofit a preexisting imaging system.

In a higher-throughput clinic deployment or hospital deployment, the remote operator interface 110 can present concurrent session status for a plurality of imaging sites 102, and the cloud-hosted orchestration and governance component 104 can maintain separate control sessions and separate immutable event records 162 for each imaging site 102. A first imaging site 102 can be in a calibration workflow while a second imaging site 102 is in a setup workflow and a third imaging site 102 is awaiting release of a pre-exposure authorization after completion of the stabilization period 160. The remote operator interface 110 can therefore support multiple virtual imaging rooms corresponding to actual x-ray rooms, clinic rooms, or portable systems without giving the on-site participant 124 at any imaging site 102 direct exposure authority.

The OEM imaging system 108 can be delivered with a transport case, a support stand, a detector, a source mount, shielding accessories, or other hardware used for off-site radiography. The on-site interaction interface 112 can present guided instructions for assembling those components, and the local state reporter 154 can verify power, detector presence, or other readiness conditions after assembly. A transported configuration can therefore preserve a consistent remote-control model even when the OEM imaging system 108 moves among homes or temporary sites.

The distributed radiography orchestration system 100 can also support optional AI-enabled analytics or machine-learning-enabled tools at the cloud-hosted orchestration and governance component 104. Such AI-enabled tools can include image-quality review, misalignment detection, protocol verification, or another post-capture or pre-authorization evaluation. The AI-enabled tools can operate on radiographic image data, on wide-field camera data, on narrow-field camera data, on current imaging parameter information, or on combinations of such inputs. The AI-enabled tools can return a recommendation, an alert, or an informational display to the remote operator interface 110 without transferring exposure authority away from the remote operator 122.

The disclosed arrangements can therefore be used with portable hardware, installed x-ray-room hardware, retrofit hardware, or mixed OEM environments while preserving the same core control model. The cloud-hosted orchestration and governance component 104 can authenticate the remote operator 122, the on-site integration system 106 can exchange command or status transactions with the OEM imaging system 108 through the OEM-defined interface 144, the remote operator interface 110 can present device status information and current imaging parameter information including site-established acquisition parameters, the on-site participant 124 can handle physical setup and communications through the on-site interaction interface 112, and the network quality gate 126 can withhold a pre-exposure authorization until measured communication conditions satisfy the authorization condition after the stabilization period 160. The distributed radiography orchestration system 100, when considered with respect to the exposure-authorization functions of the network quality gate 126, the immutable event store 128, and the communication-quality state 158, can also be referred to as a distributed radiography exposure-authorization system.

What is claimed is:

1. A method of remotely controlling a radiographic examination, the method comprising:
authenticating, at a cloud-hosted orchestration and governance component, a remote operator for a control session associated with an OEM imaging system located at an imaging site;
receiving, at the cloud-hosted orchestration and governance component and from an on-site integration system coupled to the OEM imaging system, device status information and current imaging parameter information representing a readiness state of the OEM imaging system;
presenting, by a remote operator interface coupled to the cloud-hosted orchestration and governance component, the device status information and the current imaging parameter information to the remote operator;
receiving, at the cloud-hosted orchestration and governance component, an exposure authorization from the remote operator;
transmitting, responsive to the exposure authorization and subject to one or more system-level conditions via a secure network connection through the on-site integration system, an exposure command to the OEM imaging system; and
receiving, via the secure network connection, a radiographic image captured by the OEM imaging system, wherein an on-site participant physically positions the OEM imaging system at the imaging site, and wherein an on-site interaction interface available at the imaging site lacks authority to initiate emission of X-ray radiation.

2. The method of claim 1, wherein the current imaging parameter information includes acquisition parameter information established at the imaging site, the acquisition parameter information including one or more of clinical protocol, source-to-image distance information, geometry information, mAs information, tube-voltage information, or body-size information.

3. The method of claim 1, wherein the on-site interaction interface presents guided instructions or communications for positioning the OEM imaging system, performing a calibration routine, or otherwise coordinating the radiographic examination, the communications including at least one of voice, video, or chat.

4. The method of claim 1, further comprising directing placement of a calibration phantom at the imaging site and receiving a test exposure generated with the calibration phantom before patient imaging.

5. The method of claim 1, further comprising receiving, at the cloud-hosted orchestration and governance component, image data from at least one of a wide-field camera, a narrow-field camera, or a camera integrated into the OEM imaging system when available for alignment review by the remote operator.

6. The method of claim 1, further comprising writing an immutable event record to an immutable event store, the immutable event record representing at least one of the device status information, the current imaging parameter information, the exposure authorization or evaluation of system-level conditions used to permit or inhibit transmission of the exposure command.

7. The method of claim 1, wherein the exposure authorization received from the remote operator is evaluated in conjunction with one or more additional inputs, including system state information or communication quality indicators, to determine whether exposure execution is permitted.

8. A distributed radiography orchestration system comprising:
a cloud-hosted orchestration and governance component;
a remote operator interface communicatively coupled to the cloud-hosted orchestration and governance component and configured to present device status information and current imaging parameter information to a remote operator;
an on-site integration system configured to couple the cloud-hosted orchestration and governance component to an OEM imaging system at an imaging site, the on-site integration system comprising an OEM abstraction layer configured to exchange command or status transactions with the OEM imaging system through an OEM-defined interface; and
an on-site interaction interface configured to assist an on-site participant with physical setup of the OEM imaging system, wherein the cloud-hosted orchestration and governance component is configured to issue an exposure authorization for the OEM imaging system only after receiving an exposure authorization from the remote operator, and wherein the on-site interaction interface lacks authority to initiate emission of X-ray radiation.

9. The distributed radiography orchestration system of claim 8, wherein the on-site integration system comprises a secure communication endpoint configured to establish an encrypted communication session with the cloud-hosted orchestration and governance component.

10. The distributed radiography orchestration system of claim 8, wherein the OEM abstraction layer is configured to translate a cloud-originated command into an OEM-defined interface transaction for the OEM imaging system.

11. The distributed radiography orchestration system of claim 8, wherein the on-site integration system comprises a local execution interface mechanism including at least one of a digital interface, a wireless accessory interface, a physical hand-switch interface, or an intermediary hardware adapter.

12. The distributed radiography orchestration system of claim 8, wherein the on-site integration system comprises an image and status relay and a local state reporter configured to transmit the device status information and the current imaging parameter information from the OEM imaging system to the cloud-hosted orchestration and governance component.

13. The distributed radiography orchestration system of claim 8, wherein the on-site integration system comprises a camera interface module configured to receive image data from at least one of a wide-field camera, a narrow-field camera, or a camera integrated into the OEM imaging system when available.

14. The distributed radiography orchestration system of claim 8, wherein the remote operator interface is configured to present concurrent session status for a plurality of imaging sites.

15. A distributed radiography exposure-authorization system comprising:
a cloud-hosted orchestration and governance component;
an on-site integration system communicatively coupled to an OEM imaging system;
a network quality gate communicatively coupled between the cloud-hosted orchestration and governance component and the on-site integration system; and
an immutable event store storing immutable event records, wherein the network quality gate is configured to receive objective system-measured indicators for a communication path between the cloud-hosted orchestration and governance component and the on-site integration system, determine a communication-quality state from the objective system-measured indicators, enforce a stabilization period before release of a pre-exposure authorization to the on-site integration system, write at least one of the immutable event records to the immutable event store, and withhold the pre-exposure authorization while the communication-quality state fails an authorization condition.

16. The distributed radiography exposure-authorization system of claim 15, wherein the on-site integration system is configured to transition from a primary communication interface to a fallback communication interface while the network quality gate withholds the pre-exposure authorization until completion of the stabilization period.

17. The distributed radiography exposure-authorization system of claim 15, wherein the objective system-measured indicators comprise at least one of latency values, jitter values, packet-loss values, or connection-continuity values for the communication path.

18. The distributed radiography exposure-authorization system of claim 15, wherein the immutable event records store the communication-quality state, the authorization condition, and issuance or withholding of the pre-exposure authorization.

19. The distributed radiography exposure-authorization system of claim 15, wherein the on-site integration system is configured to block the pre-exposure authorization when an on-site participant proximity measurement indicates that an on-site participant is within a local safety boundary around the OEM imaging system.

20. The distributed radiography exposure-authorization system of claim 15, wherein the network quality gate is limited to pre-exposure authorization and does not participate in radiation termination after initiation of an authorized exposure.
